(19) United States
(12) Patent Application Publication
(10) Pub. No.: US 2026/0026705 A1
(43) Pub. Date: Jan. 29, 2026

(54) SYSTEM AND METHOD FOR QUANTITATIVELY MEASURING DYSPNEA

(71) Applicant: CereVu Medical, Inc., San Francisco, CA (US)

(72) Inventor: Alireza Akhbardeh, Daly City, CA (US)

(73) Assignee: CereVu Medical, Inc., San Francisco, CA (US)

(21) Appl. No.: 19/229,091

(22) Filed: Jun. 5, 2025

Related U.S. Application Data
(63) Continuation of application No. 17/534,160, filed on Nov. 23, 2021, now abandoned.
(60) Provisional application No. 63/117,952, filed on Nov. 24, 2020.

(51) Int. Cl. A61B 5/08; A61B 5/00; A61B 5/01; A61B 5/024; A61B 5/053; A61B 5/145; G16H 50/30

(52) U.S. Cl. CPC A61B 5/0816; A61B 5/01; A61B 5/024; A61B 5/053; A61B 5/0823; A61B 5/14542; A61B 5/6814; A61B 5/6822; A61B 5/7275; G16H 50/30; A61B 2562/0219

(57) ABSTRACT

A system for quantitatively measuring dyspnea experienced by a person includes a hemodynamic sensor system configured to be held in close proximity to, or in contact with, the person's head or neck, and measure hemodynamic parameters of the person. The system includes a respiratory sensor system to measure respiratory metrics of the person, and a data processor configured to communicate with the hemodynamic sensor system to receive the hemodynamic parameters and with the respiratory sensor system to receive the respiratory metrics. The data processor is configured to calculate, based on the hemodynamic parameters, a quantitative score to represent physical discomfort experienced by the person, and make a determination, based on the quantitative score and the respiratory metrics, that the physical discomfort is caused by dyspnea rather than physical pain. The determination includes a sub-determination that the respiratory metrics have changed by a particular amount within a particular period of time.

This application is a continuation of U.S. application Ser. No. 17/534,160, filed Nov. 23, 2021, which claims priority to U.S. Provisional Application No. 63/117,952, filed Nov. 24, 2020, which are incorporated herein by reference in entirety.

### BACKGROUND

### 1. Technical Field

Currently claimed embodiments of the invention relate to measurement of dyspnea.

### 2. Discussion of Related Art

Pandemic viral pneumonias such as COVID-19 have resulted in significant stress on the U.S. healthcare system as hospitals face shortages of resources and staff. For instance, almost 80% of the patients who test positive for COVID-19 have mild and moderate symptoms and therefore do not require immediate care¹. Approximately 15% of patients are severely afflicted and require respiratory support with oxygen, postural maneuvers, and high flow therapy, while 5% become critically ill and require mechanical ventilatory support. Likewise, patients suffering from severe asthma or chronic obstructive pulmonary disease (COPD) also often require ventilation and sedation. Ventilated patients are heavily anesthetized with sedation and pain management drugs in order to manage anxiety and the pharyngeal reflex (gagging response) of intubation.

Although it is easier to manage a patient under heavy sedation (no patient response or resistance), there are multiple side-effects and adverse events. Heavily sedated patients are at risk of long-term cognitive impairment and executive function². Up to 60% of heavily sedated ICU patients experience gastrointestinal dysmotility³. This can lead to poor nutritional uptake, thus interfering with recovery. Heavy sedation reduces phrenic nerve activation of the diaphragm, leading to diaphragm muscle atrophy and increased time weaning patients from the ventilator. Untold consequences include increased length of stay, ventilator shortages, ventilator associated nosocomial pneumonia, neurocognitive dysfunction and post-traumatic stress disorder. Patients with compromised lungs, such as COPD, asthma, and viral pneumonia patients that suffer from dyspnea (shortness of breath), are subjected to increased positive pressure and acute lung injury from associated barotrauma. Barotrauma contributes to significant increases in length of stay, long-term disability and mortality. Inadequate pain management is common in ventilated patients. 71% of patients recall excess pain during their ICU stay⁴, increasing the risk of post-traumatic stress disorder. Shortening ventilation time would improve patient outcomes, with the added benefit of alleviating ventilator and medical supply shortages during a pandemic⁵ such as COVID-19.

The mortality rate for viral pneumonia patients that require mechanical ventilation is up to two times the rate of patients suffering from other types of viral pneumonia⁶. A critical unmet need exists to improve respiratory monitoring and tailor the treatment strategy to the needs of these patients. There are approximately five million ICU patients a year in the United States⁷ and over 20% require mechanical ventilation at some time during their stay⁸. The surge population associated with some viral diseases such as COVID-19 patients is estimated to be much higher.

Furthermore, it has become clear that a greater long-term need is to use remote patient monitoring (RPM) for long-standing respiratory distress diseases and disorders since the markets are very large and non-cyclical. Patients with respiratory-distressed illnesses like chronic obstructive pulmonary disease (COPD), asthma, and pneumonia would benefit from monitoring of their critical biomarkers to ensure they receive appropriate and timely treatment. Although monitoring and treatment of these patients are usually executed effectively in hospital settings, they have a high hospital-readmission rate without the same level of post-discharge monitoring. The need for more effective remote patient monitoring and management has been exacerbated by the current COVID-19 pandemic as patients inundated emergency rooms.

There are many companies with physiological data and patient bedside monitoring products on the market. However, there are no FDA approved products for assessing dyspnea.

The ability to measure dyspnea as well as the depth of sedation would allow clinicians to optimize levels of sedation while decreasing complications and shortening periods on mechanical ventilation. The ability to control dyspnea but minimize sedation while still providing pain relief would potentially reduce ventilation-associated complications and improve the ventilation weaning process. These abilities would address a significant problem associated with ventilation in all chronic patients.

### SUMMARY

A system for quantitatively measuring dyspnea being experienced by a person, comprising a hemodynamic sensor system configured to be held in close proximity to, or in contact with, one of said person's head and said person's neck, said hemodynamic sensor system configured to provide measurements of hemodynamic parameters of said person. The system comprises a respiratory sensor system configured to provide measurements of respiratory metrics of said person and a data processor configured to communicate with said hemodynamic sensor system to receive said measurements of hemodynamic parameters and configured to communicate with said respiratory sensor system to receive said measurements of respiratory metrics. The data processor is further configured to calculate, based on the measurements of hemodynamic parameters, a quantitative score to represent a state of physical discomfort experienced by the person, and make a determination, based on the quantitative score and the measurements of respiratory metrics, that the state of physical discomfort experienced by the person is caused by dyspnea. The determination comprises a sub-determination that the respiratory metrics have changed by a particular amount within a particular period of time.

A non-transitory machine-readable medium storing a program for quantitatively measuring dyspnea being experienced by a person, which when executed by a data processor configures said data processor to receive measurements of hemodynamic parameters of said person, from a hemodynamic sensor system held in close proximity to, or in contact with, one of said person's head and said person's neck, receive measurements of respiratory metrics of said person from a respiratory sensor system, calculate, based on the measurements of hemodynamic parameters, a quantitative score to represent a state of physical discomfort experienced by the person, and make a determination, based on the quantitative score and the measurements of respiratory metrics, that the state of physical discomfort experienced by the person is caused by dyspnea. The determination comprises a sub-determination that the respiratory metrics have changed by a particular amount within a particular period of time.

A method for quantitatively measuring dyspnea being experienced by a person, comprising receiving measurements of hemodynamic parameters of said person, from a hemodynamic sensor system held in close proximity to, or in contact with, one of said person's head and said person's neck, receiving measurements of respiratory metrics of said person from a respiratory sensor system, calculating, based on the measurements of hemodynamic parameters, a quantitative score to represent a state of physical discomfort experienced by the person, and making a determination, based on the quantitative score and the measurements of respiratory metrics, that the state of physical discomfort experienced by the person is caused by dyspnea. The determination comprises a sub-determination that the respiratory metrics have changed by a particular amount within a particular period of time.

### BRIEF DESCRIPTION OF THE DRAWINGS

Further objectives and advantages will become apparent from a consideration of the description, drawings, and examples.

FIG. 1 shows a top view for a wearable device of some embodiments.

FIG. 2 shows a CO₂ test of some embodiments to simulate dyspnea.

FIGS. 3A and 3B show the general pipeline of real-time tracking of cerebral hemodynamic response (RTCHR).

FIG. 4 illustrates an example of a pulse oximetry signal.

FIG. 5 shows a ratio that is used as the input to a “lookup table” used in some embodiments to obtain the SpO₂ value.

FIG. 6 shows an example of detection of envelope of signal using secant angle.

FIG. 7 shows a beat-to-beat heart and respiration rate calculation schema with a modified Hamilton algorithm of some embodiments.

FIG. 8 illustrates a pipeline to predict hemodynamic changes using a predictive model.

FIGS. 9A, 9B, and 9C illustrate different behaviors for different locations of the pole.

FIG. 10 shows the rough estimate arrival of onset of dyspnea, pain, trauma, or unconsciousness arrival by use of average and/or max of poles magnitudes.

FIG. 11 illustrates ventilated patient challenges for viral pneumonia disease.

FIG. 12 shows receiver operating characteristic (ROC) curves and corresponding area under curve for healthy volunteers and a woman in labor.

FIGS. 13A, 13B, 13C, and 13D show routine vital signs and the dyspnea scoring index for data with breath holding every two minutes.

FIGS. 14A, 14B, 14C, and 14D show the dyspnea scoring index and routine vital signs for data with breathing to a bag for two minutes.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed, and other methods developed without departing from the broad concepts of the current invention. All references cited anywhere in this specification, including the Background and Detailed Description sections, are incorporated by reference as if each had been individually incorporated.

The Center for Disease Control (CDC) guidelines recommend monitoring dyspnea (shortness of breath) as a major indicator of increased criticality, for example in viral pneumonia cases such as COVID-19, patients with severe asthma, and patients with COPD. The level of dyspnea, or air hunger, does not always correlate with oxygen saturation^{9,10}. Conversely, a patient can be hypoxemic, even if the patient is not particularly short of breath. The respiratory system and pulmonary function are not the only factors impacting respiratory outcomes. Other factors such as cardiovascular health, level of conditioning (or deconditioning), medications, emotional state, and weather also impact the quality of breathing. For patients with dyspnea but a normal oxygen saturation (SpO₂), supplemental oxygenation will not mitigate risk of respiratory progression.

The proposed method of some embodiments calculates a novel biomarker that objectively measures dyspnea in real-time by utilizing cerebral hemoglobin oxygenation and hemodynamic changes measured using optical spectrometry technology. This biomarker is computed in some embodiments using signal processing and predictive model algorithms. The biomarker is displayed in some embodiments as a single number or score to detect and quantify dyspnea and distinguish dyspnea from other hemodynamic responses including physical pain which is carried along similar brain pathways to dyspnea¹¹. For example, the biomarker is calculated in some embodiments using a hemodynamic predictive model which is described in more detail in U.S. Publication No. 2014/0296655 (Oct. 2, 2014), incorporated herein by reference, and below in the appendix section entitled “Real-time hemodynamic response changes tracking scheme.”

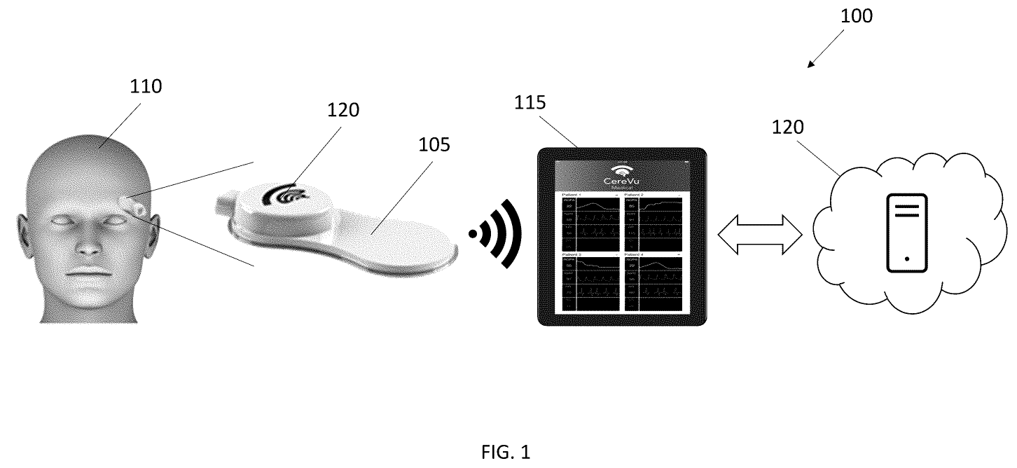

As illustrated by example in FIG. 1, the biomarker can be measured using a system 100 in some embodiments that is capable of measuring parameters for vital signs and bodily functions including, but not limited to, SpO₂/hypoxia, blood oxygenation, bio-potentials such as electrocardiograms, respiration rate, heart rate, body temperature, body posture and activities. In some embodiments, the system 100 extracts all parameters from a wearable patch device 105 (which could be worn anywhere on the head or neck, for example, the forehead, the car, etc.) that includes electrodes and/or optical sensors. In some embodiments the system 100 also possesses other sensors, including but not limited to accelerometers/gyroscopes, impedance sensors, surface electrodes, temperature sensors, and microphones. In some embodiments the uses some or all of these parameters, as measured by some or all of these sensors, to measure nociception (objective pain) and dyspnea. In some embodiments, the system collects data and computes the biomarker in real-time.

A top view of a small, low-cost wearable patch device 105 of some embodiments is shown in FIG. 1 that captures key vital signs as well as novel biomarkers to objectively measure dyspnea and nociceptive pain (hemodynamic response) using a wearable patch (e.g., on a patient's head 110) and a display 115 (e.g., a tablet or computer monitor). In some embodiments, the device also has a wireless communication module 120 to wirelessly communicate with the display 115. In some embodiments, the display 115 is a mobile application that also serves as a user interface for viewing, logging and playing back data. In some embodiments, the mobile application pushes/pulls data to a cloud-based hub 120 for centralized monitoring.

In some embodiments, the proposed system and method employ the operating principle of multi-distance diffuse reflectance spectroscopy. In some embodiments, the system uses near-infrared (NIR), infrared (IR), and RED spectroscopy to sample a cross-section of tissue microvasculature (a mixed bed of arterioles, capillaries and venules) in brain tissue and analyzes the light returned after the blood has passed through the tissue. The spectroscopic analysis determines changes in the concentrations of hemoglobin in its oxygenated and deoxygenated states. In some embodiments, the system calculates an index score (hereafter referred to as the Hm index) based on the trended change in regional hemoglobin oxygenation and nociceptive hemodynamic response in the sampled tissue.

In some embodiments, the Hm index is calculated using only measurements and data from an optical sensor. In some embodiments, additional sensors are used to reduce false positives, identify motion artifacts, and validate the index. These other supplementary sensors include but are not limited to accelerometers, gyroscopes, impedance sensors, surface electrodes, temperature sensors, and microphones.

For example, in some embodiments, an accelerometer is used to identity motion artifacts and void false positives. In some embodiments, skin impedance measurements from an impedance sensor are used to make sure that the path is not loose, and that the optical data is valid. In some embodiments, bio-potential (e.g., measured by a surface electrode) and body temperature (e.g., measured by a temperature sensor) are used to improve pain estimation. In some embodiments, vital signs such as heart rate, respiration rate, and SpO₂ are extracted from a surface electrode or an optical sensor.

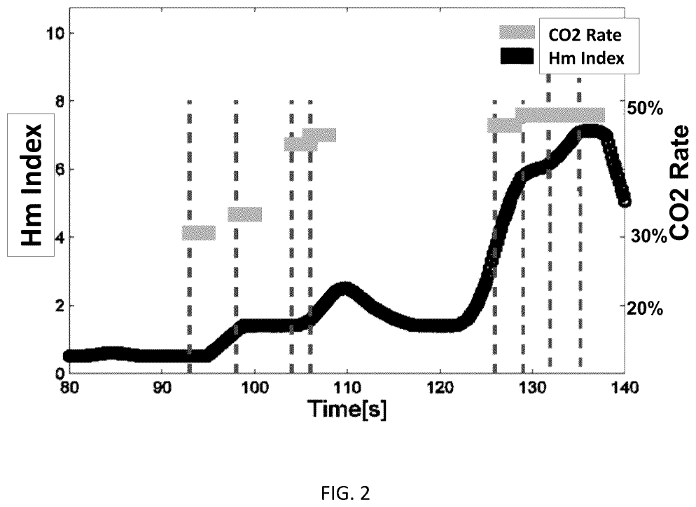

Dangers at el.¹¹ reported that dyspnea and pain share are sensations that converge of similar neural networks in the brain. They also concluded that air hunger interferes with the cortical mechanisms responsible to painful laser skin stimulation. This hypothesis was validated in the present work by simulating a dyspnea CO₂ test in the absence of a physical pain stimulus. In this experiment, CO₂, SpO₂, heart rate, systolic & diastolic blood pressures were monitored using standard commercial device. Similar to the Dangers at el. experiment, the CO₂ rate was limited to 50% to avoid a contribution of hypoxia to dyspnea and air hunger. CO₂ increase was conducted twice to induce dyspnea as shown in FIG. 2. This shows SpO₂ and CO₂ rate are necessarily correlated. This re-validates Dangers at el. findings: “dyspnea and pain share are sensations that converge of similar neural networks in the brain”. This also confirms that level of dyspnea, or air hunger, does not always correlate with oxygen saturation.

FIG. 2 shows a CO₂ test to simulate dyspnea: the dyspnea index, a novel nociceptive biomarker called “Hm Index” (in black) vs. CO₂ rates (levels in green—the amount of CO₂ gas in a person's blood). CO₂ was increased twice to trigger dyspnea. Continuously monitored CO₂ rate was controlled to not exceed 50% to avoid a contribution of hypoxia (absence of enough oxygen measured by SpO₂) to dyspnea. Dynamic range for Hm index is 0 to 10. Vertical dot lines shows time points that logged CO₂ rates and vital signs as following: (CO₂ rate=35, BP=117/73, SpO₂=98), (CO₂ rate=38, BP=119/80, SpO₂=92), (CO₂ rate=48, BP=119/80, SpO₂=96), (CO₂ rate=50, BP=119/80, SpO₂=94), (CO₂ rate=50, BP=140/87, SpO₂=82), (CO₂ rate=50, BP=140/87, SpO₂=99), respectively for time 93, 98, 106, 124, & 137 seconds.

To distinguish pain and dyspnea using an index such as the Hm index, changes in respiratory vital metrics such as respiration rate and SpO₂ are used in some embodiments. The rise in respiratory rate increases the work of breathing, and the obstructed outflow of air in COPD leads to worsening hyperinflation and heightened stimulation of mechanoreceptors. This results in more dyspnea, which may make the patient breath faster and worsen the hyperinflation still more 12.13.

If significant changes are observed in respiratory metrics, the Hm index indicates dyspnea and not pain. Otherwise, the Hm index indicates pain and not dyspnea. In other words, for pain, dyspnea is a false positive, and for dyspnea, pain is a false positive. By observing changes in respiratory metrics, what the Hm index indicates for can be identified, and false positives eliminated.

Multiple cardiovascular events other than dyspnea could cause changes in respiratory metrics and they alone cannot be used as biomarkers for dyspnea, however these events along with the Hm index along with the respiratory metrics precisely quantify dyspnea in some embodiments.

The following describes some specific examples according to some embodiments of the current invention. The general concepts of this invention are not limited to these particular examples.

### Real-Time Hemodynamic Response Changes Tracking Scheme

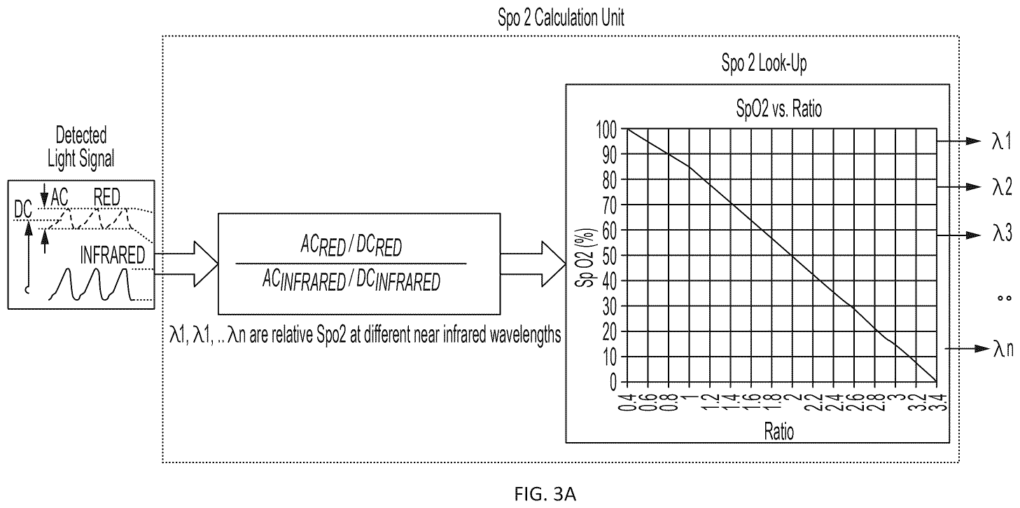

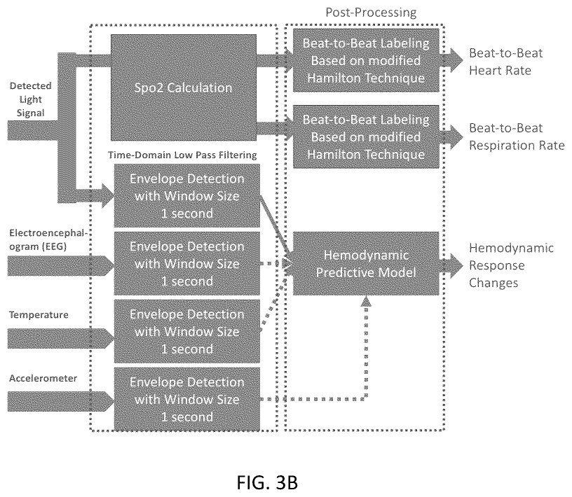

The real-time tracking of cerebral hemodynamic response (RTCHR) system and method consists of several steps and processing units to calculate several hemodynamic parameters including 1) heart (pulse) rate, 2) respiration rate and 3) hemodynamic response to an internal and/or external stimulus. FIGS. 3A and 3B show the general pipeline of real-time hemodynamic response changes tracking (RTCHR). FIG. 3A shows the oxygen saturation (SpO₂) calculation, and FIG. 3B shows Pre and Post Processing steps to calculate heart/respiration rates and estimate hemodynamic response changes in real-time.

### SpO₂ Calculation Unit

Optical sensing is a non-invasive measurement of the oxygen saturation (SpO₂). Oxygen saturation is defined as the measurement of the amount of oxygen dissolved in blood, based on the detection of Hemoglobin and Deoxyhemoglobin.

Two or more different light wavelengths are used in some embodiments to measure the actual difference in the absorption spectra of HbO2 and Hb. The bloodstream is affected by the concentration of HbO2 and Hb, and their absorption coefficients are measured using red light and infrared light spectra. A pulse oximeter is an optical sensing-based device which uses two wavelengths, of red light spectra and infrared light spectra. As a non-limiting example, in some embodiments, a wavelength of 660 nm may be used for the red light spectra, and a wavelength of 940 nm may be used for the ifrared light spectra. In other embodiments, other wavelengths may be used. Deoxygenated and oxygenated hemoglobin absorb different wavelengths. Deoxygenated hemoglobin (Hb) has a higher absorption at red light spectra and oxygenated hemoglobin (HbO2) has a higher absorption at infrared light spectra.

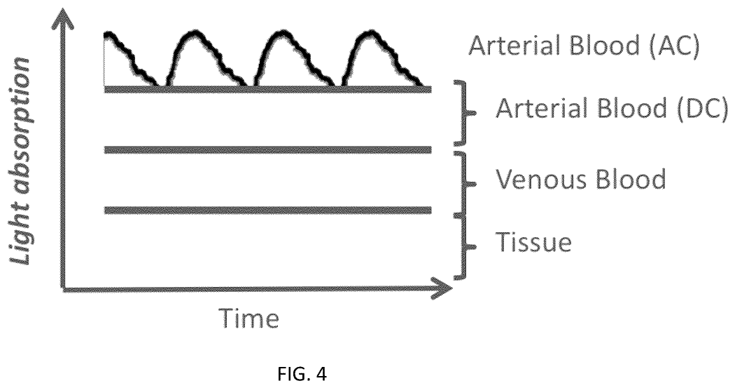

A photodetector in the sensor perceives the non-absorbed light from the LEDs. This signal is inverted using hardware with operational amplifiers in some embodiments, and the result is a signal like the one in FIG. 4, which shows a light absorption diagram and a typical pulse oximetry signal. This signal represents the light that has been absorbed by the tissue and is divided in a DC component and an AC component. The DC component represents the light absorption of the tissue, venous blood, and non-pulsatile arterial blood. The AC component represents the pulsatile arterial blood.

(I_{ac}) λ_{RED}=Light intensity at red light spectra and (I_{ac}) λ_{INFRARED}=Light intensity at infrared light spectra, where only the AC level is present. An example of a pulse oximetry signal is represented in FIG. 4. The signal represents the pulsatile arterial blood absorption. The beats per minute can be calculated using this signal.

To perform the oxygen saturation (SpO₂) calculation, in some embodiments SpO₂ is taken out from a table stored on the memory calculated with empirical formulas out of so-called absorption ratio as defined below from two or more wavelengths from the pulsatile-added volume of oxygenated arterial blood (AC/DC), using Equation (1):

$$\frac{{AC}_{RED} / {DC}_{RED}}{{AC}_{INFRARED} / {DC}_{INFRARED}} \qquad ( 1 )$$

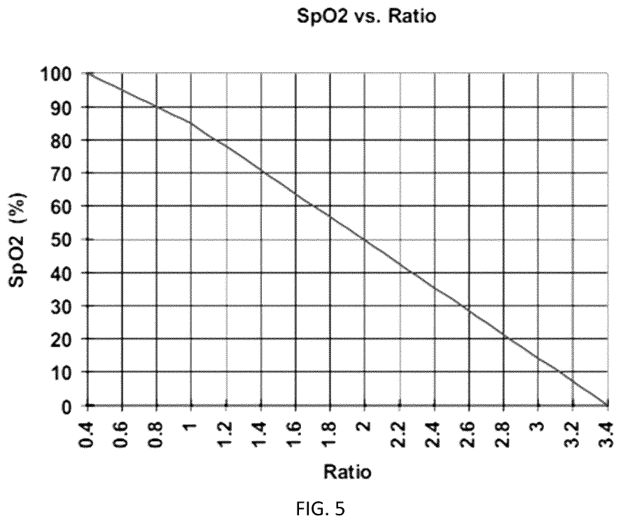

SpO₂ is determined in some embodiments from a lookup table stored in memory and calculated with empirical formulas (see FIG. 5). Using the example lookup table of FIG. 5, a ratio of 1 represents a SpO₂ of 85%, a ratio of 0.4 represents SpO₂ of 100%, and a ratio of 3.4 represents SpO₂ of 0%. For more reliability, the table must be based on experimental measurements of healthy patients.

FIG. 5 shows the ratio that is used as the input to a “lookup table” used in some embodiments. The lookup table of FIG. 5 is just one example of possible lookup tables that may be used, after calculation from empirical data. The SpO₂ value is the result of the “lookup” function.

In other embodiments, SpO₂ is calculated from the Beer-Lambert law by taking the AC component of only the signal, and determining the ratio by using Equation (2). SpO₂ is the value of R×100:

$$R = \frac{{log}_{10} ( {I}_{ac} ) {λ}_{1}}{{log}_{10} ( {I}_{ac} ) {λ}_{2}} \qquad ( 2 )$$

In Equation (2), (lac) λ_{RED} is the light intensity at red light spectra and (I_{ac}) λ_{INFRARED} is the light intensity at infrared light spectra, where only the AC level is present. A typical pulse oximetry signal is represented in FIG. 4. The signal represents the pulsatile arterial blood absorption. The beats per minute can be calculated using this signal.

### Envelope Detection Based Time-Domain Low-Pass Filtering

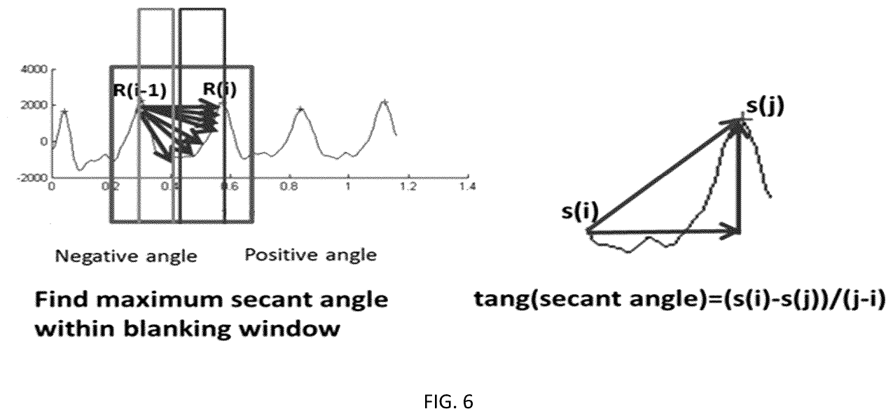

To detect upper and lower envelopes of the raw optical data, the secant angle is used in some embodiments. This smooths the data and eliminates carrier frequencies which are used to emit LEDs in different wavelengths. For Upper envelope, the detector searches for maximum secant angle within a user defined blanking window. For lower envelope, it searches for minimum secant angle within the blanking window. Data samples for the upper envelope presents the timing of R-wave locations, while data samples for the lower envelope presents the timing of S-wave locations. FIG. 6 illustrates how the envelope of a signal can be calculated using secant angles in some embodiments. For Upper envelope detector, it searches for the maximum secant angle within a user defined blanking window. For lower envelope it searches for the minimum secant angle within the blanking window.

SpO₂ Pulse Annotation Optical signals (red and IR wavelength data) to Calculate Beat-To-Beat Heart/Respiration Rates

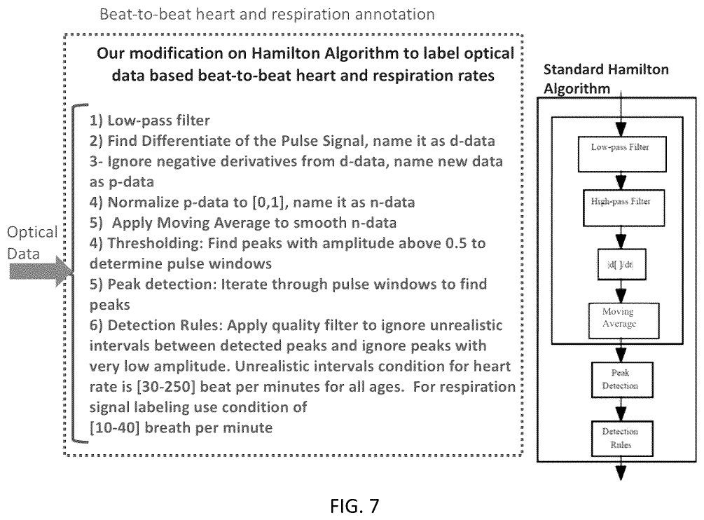

An approach in some embodiments to annotate electrocardiogram (ECG) data is the Hamilton technique. To calculate beat-to-beat heart/respiration rates, some embodiments improve upon the Hamilton algorithm, as illustrated in FIG. 7. FIG. 7 shows the beat-to-beat heart and respiration rate calculation schema with modified Hamilton algorithm.

### Hemodynamic Predictive Modeling

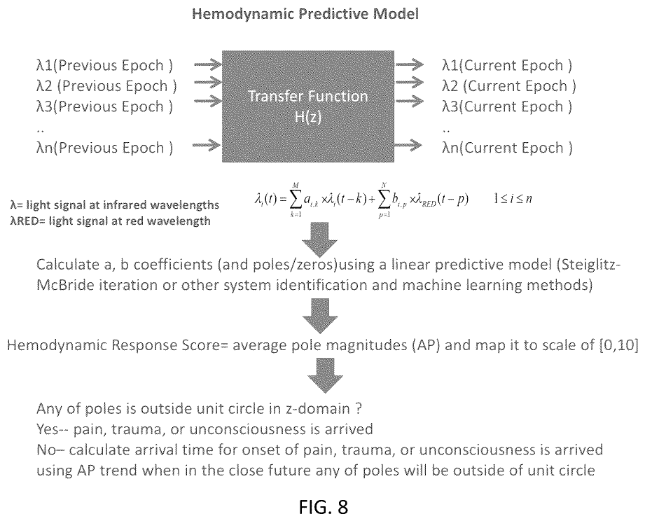

To predict hemodynamic changes, some embodiments use a predictive model shown in FIG. 8, which is described below. The system in FIG. 8 corresponds to Equation (3):

$${λ}_{i} ( t ) = {\sum }_{k = 1}^{M} {a}_{i , k} \times {λ}_{i} ( t - k ) + {\sum }_{p = 1}^{N} {b}_{i , p} \times {λ}_{RED} ( t - p ) , 1 \leq i \leq n \qquad ( 3 )$$

where aᵢ and bᵢ are the weights with which each light signal at red and infrared wavelength, respectively, contributes to the hemodynamic history, and where M and N are the number of light signal samples included in the hemodynamic history.

In some embodiments, artificial intelligence and system identification methods are used to determine the parameters for Equation (3). In some embodiments, the values of M, N, aᵢ, and bᵢ (where i=1 . . . . M) are determined using a linear predictive model called linear model using Steiglitz-McBride iteration in some embodiments. In the z-domain, a system is represented as a transfer function. The transfer function H (z) representing Equation (3) in the z-domain is represented by Equation (4):

$$H ( z ) = {\sum }_{i = 1}^{M} \frac{{A}_{i}}{z - {α}_{i}} \qquad ( 4 )$$

where Aᵢ and αᵢ (i=1 . . . . M) are parameters derived from aᵢ, bᵢ (i=1 . . . . M), and Mis the number of components in H(z), which is also the number of light signal samples in the hemodynamic history. In Equation (4), when z is set equal to αᵢ, one obtains a pole of the system; a system has as many poles as there are values of a (i.e., M). According to the z-domain stability theory (bounded-input-bounded output concepts in electrical engineering) the system is unstable if a pole falls outside of the unit circle (|z|=1, see FIG. 8).

FIG. 8 shows a pipeline to predict hemodynamic changes using a predictive model.

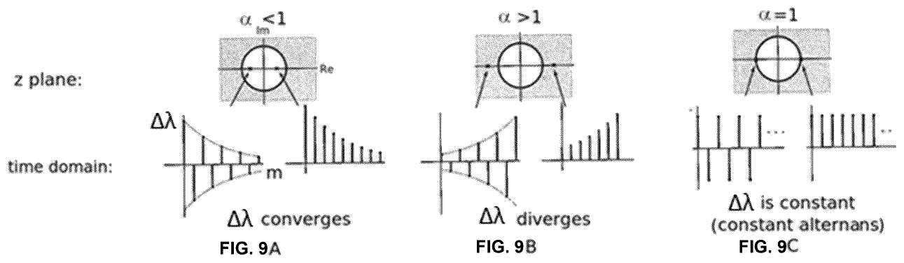

FIGS. 9A-9C illustrate the different behaviors of the system for different locations of the pole. In FIG. 9A, poles located within the unit circle (α<1) result in decreasing magnitude of the oscillations in light signal at infrared wavelengths (Δλ) following a perturbation of the system (a premature or a delayed beat). In FIG. 9B, where α>1, the magnitude of the oscillations caused by the perturbation increases. As mentioned above, the z-transform decomposes a system in the time domain into a series of power components with different bases. In the time domain, the unit circle (|z|=1) corresponds to a power component with a base of 1. A power component diverges when its base is bigger than 1 and converges if the base is smaller than 1. Computationally efficient implementations of stability analysis in z-domain are available, enabling real-time execution of the method in the clinic.

FIGS. 9A-9C illustrate different system behaviors for different locations of the pole α. The location of the pole is shown in the z-plane, with the real (Re) and imaginary (Im) values of z being on the horizontal and vertical axes, respectively. The radius of the circle in the z-plane is 1 (unit circle). In the time domain, the behavior of the system is illustrated by the decreasing (FIG. 9A), increasing (FIG. 9B), or constant (FIG. 9C) magnitude of the oscillations following a perturbation (a premature or a delayed beat), depending on the location of a in the z-plane (inside, outside, or at the boundary of, the unit circle). m is the beat number, counted from the onset of perturbation.

To score hemodynamic response (e.g., to calculate the Hm index in some embodiments) some embodiments use an average of poles' magnitudes, scaled from 0 to 10. In the objective pain assessment, the hemodynamic response score has the following interpretation:
0-1: No Pain
2-3: Mild Pain
4-5: Moderate Pain (discomforting)
6-7: Severe Pain (distressing)
8-9: Very Severe Pain (intense)
10: Unbearable Pain

In the level of consciousness assessment, the hemodynamic response score has the following interpretation:
0-1: Awake
2-3: Light/Moderate Sedation
4-5: General Anesthesia
6-7: Deep Hypnotic State
8-9: Burst Suppression
10: Fully Unconscious

The magnitudes of the poles may also be a sign of onset of pain arrival. For example, if any of poles are outside the unit circle, that means onset of pain is about to start. The likelihood of pain, trauma, or unconsciousness arrival is estimated by Equation (5):

$$Likelihood = \frac{Number of poles outside unit circle}{Total number of poles} \qquad ( 5 )$$

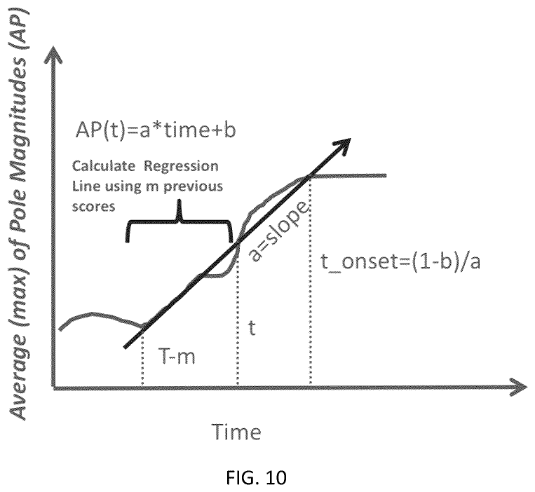

If average and/or max of poles magnitudes (AP) is gradually become closer to unit circle in z-domain space, the slope of the trend is used in some embodiments to roughly estimate arrival of unset of dyspnea, pain, trauma, or unconsciousness arrival, as illustrated in FIG. 10.

FIG. 10 shows the rough estimate arrival of onset of dyspnea, pain, trauma, or unconsciousness arrival by use of average and/or max of poles magnitudes (AP).

### Biomarker for Assessment and Communication of Optimal Sedation Levels

Some embodiments of the invention significantly improve ventilation by incorporating the dyspnea proprietary biomarker into patient monitoring that assesses and communicates optimal sedation levels. This is accomplished in some embodiments by merging the novel biomarker (e.g., Hm index) with respiratory biomarkers (e.g., respiration and SpO₂).

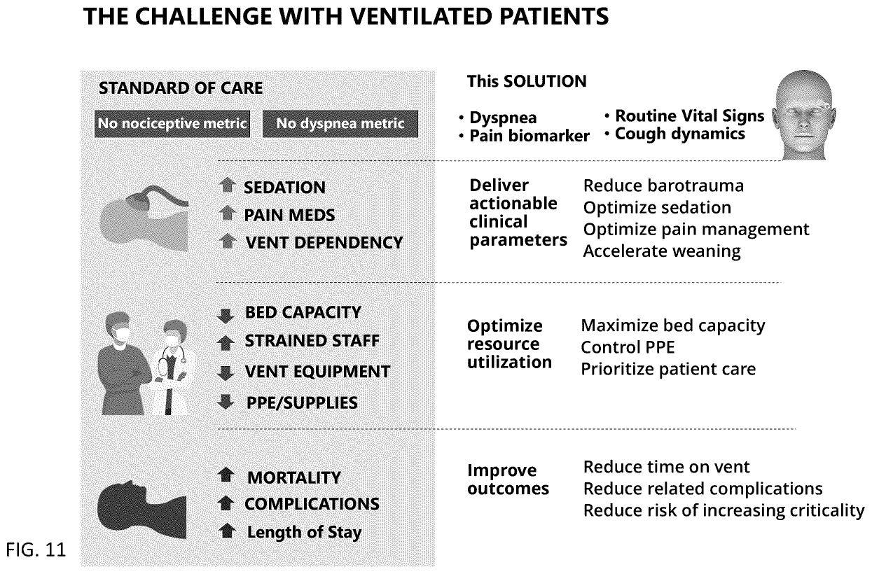

As shown in FIG. 11, some embodiments of the invention will enable clinicians to better monitor the effect of the analgesic and sedation medications allowing for optimal dosing and balancing the competing goals of minimizing discomfort and minimizing sedation. The ability to continuously monitor this new biomarker will facilitate a gradual decrease in ventilator dependency and accelerate ventilator weening as patients recover.

FIG. 11 illustrates ventilated patient challenges for pulmonary disease and ventilated patients.

Predict COPD flare-up (Exacerbation): Monitoring and predicting patient's dyspneic condition could reduce the impact of exacerbations and the risk of hospitalization. To assist clinical decision making, predictive tools have been developed over the years and is an active field. Recently, machine learning models have been developed to aggregate information from multiple sources and aid the decision making. Sanchez-Morillo et al. did an extensive literature review and survey on such predictive models²⁰. They provided a systematic review of existing algorithms and the factors associated with their performance in detecting exacerbations in COPD patients. Example of machine learning models previously used by others include support vector machines, logistic regression, Naive Bayes, K-nearest neighbors, gradient boosted and ensemble decision tree methods.

Predict the likelihood that a patient is having a COPD flare-up (exacerbation) by use objectively measuring dyspnea (described in this patent), or coughing dynamics, or other Data types.

Additionally, in some embodiments a centralized data hub will reduce physical interaction between clinicians and critically ill patients by enabling patient consolidated remote monitoring. This provides a significant value in ad hoc field hospitals where a shortage of clinicians and monitoring equipment is inevitable, and decreases risk of clinician infection.

A system 100 has been developed to capture routine vital signs as well as a novel biomarker Hm index for measuring dyspnea as well as nociception, the physiological response to unpleasant pain. Some embodiments of the system 100 include 1) a small, wearable patch 105 with multiple bio-sensors, and 2) a user interface 115 for viewing, storing, and replaying data. The system 100 pushes the data to a cloud-based hub for centralized monitoring in some embodiments, and continuously displays the patient's physiological parameters listed above in real-time.

The current standard of care is to assess a ventilated patient's pain by using subjective behavioral correlative proxies such as compliance with the ventilator, facial grimacing, body movement and body tension¹⁴. This methodology is dependent on clinician judgement and the availability to make frequent detailed assessments. In busy and understaffed ICUs, timely in-depth patient assessments are a luxury. The system of some embodiments improves respiratory monitoring of critical patients by continuously and objectively monitoring their nociceptive pain, dyspnea and hypoxia. No other monitoring system is capable of quantifying nociceptive dyspnea and pain.

The system is capable in some embodiments of measuring vital signs (SpO₂/hypoxia, respiration rate, heart rate, body temperature, body posture and activities, skin impedance). The patch of some embodiments includes some or all of a multi-frequency LED, multiple optical detectors, multiple impedance sensors, a temperature sensor, an accelerometer/gyroscope, a battery, and a Bluetooth transmitter. As a non-limiting example, in some embodiments the system uses four optical detectors and four impedance sensors. It can be used in both a wired and wireless format. Unlike wearables that measure standard vital signs, the system also measures dyspnea, coughing dynamics, and nociceptive pain. The system of some embodiments includes the wearable patch and a tablet monitor with eventual capability of merging the system's unique data into other critical care monitoring systems.

### Preliminary Data

There are no analytical tools to assess dyspnea and pain in ventilated patients. Clinicians are trained to observe proxy indicators (heart rate, respiration, facial expressions, muscle tension) for both conditions. This methodology is dependent on clinician judgement and the availability to make frequent detailed assessments. In busy and understaffed units, timely in-depth patient assessments are a luxury. The system of some embodiments will improve respiratory monitoring of critical patients by continuously and objectively monitoring nociceptive pain and dyspnea. Continuous and objective measurement of these conditions will improve outcomes, shorten the length of ventilation time, and improve ICU operations.

A top view of a small, low-cost wearable device 105 of some embodiments is shown in FIG. 1 that captures key vital signs as well as novel biomarkers to objectively measure dyspnea and nociceptive pain (hemodynamic response) using a wearable patch (e.g., on a patient's head 110) and a display 115 (e.g., a tablet or computer monitor). In some embodiments, the device also has a wireless communication module 120 to wirelessly communicate with a mobile application as well for a user interface for viewing, logging and playing back data. In some embodiments, the mobile application pushes data to cloud-based hub for centralized monitoring.

The urgent need to manage viral pneumonia diseases such as COVID-19 drives repurposing the wearable patch 105 as a patient bed-side monitor in critical care units and field-hospitals to continuously monitor viral pneumonia patients (both sedated and awake patients). This will be accomplished by continuously monitoring routine vital signs and the novel biomarkers of some embodiments; coughing dynamics, dyspnea, and nociceptive pain.

Routine Vital Signs: Absolute values and changes to body temperature, respiration rate, heart rate, and SpO₂/hypoxia will be continuously monitored in some embodiments.

Coughing Dynamics: The patch in some embodiments possesses a highly sensitive MEMS-based accelerometer, providing the ability of measuring vibrations and minute movements, thus providing the capability of detecting coughing frequency and intensity. In some embodiments the patch also includes a microphone to provide additional coughing detection capability. This ability to measure frequency of occurrence and intensity of coughs could be beneficial in evaluating patient deterioration and recovery.

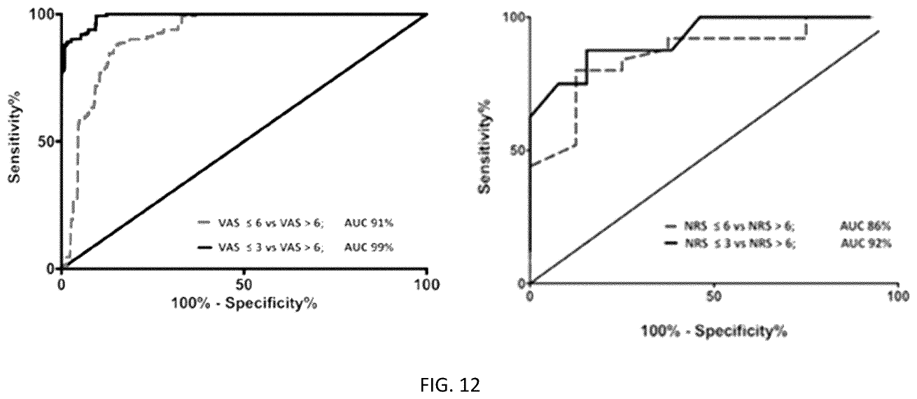

Nociceptive Pain Biomarker: The proposed nociceptive pain biomarker of some embodiments allows for the continuous monitoring of pain during the patient's ICU stay. The nociceptive pain biomarker algorithm was validated in a three-arm clinical study: child-birth procedure (OB), general anesthesia or sedated patients (GA), and healthy volunteers. The results were published in the Journal of Pain Research¹⁵. As discussed in the publication, objective pain was based on assessing changes in hemodynamic response as measured by the proposed system and it demonstrated over 90% correlation with the level of pain experienced by patients/volunteers in the two arms where subjective reporting was possible. FIG. 12 shows receiver operating characteristic (ROC) curves and corresponding area under curve for healthy volunteers and a woman in labor. Recent studies using fMRI and PET show similar results¹⁶ when measuring subjective pain to blood-oxygen-level-dependency (BOLD—a method used in functional magnetic resonance imaging to observe different areas of the brain). A review paper¹⁷ discusses the similarity between BOLD and optical imaging driven hemodynamic response.

FIG. 12 shows ROC analysis and corresponding area under curve for (left) healthy volunteers and (right) woman in labor (OBGYN). The proposed biomarker of some embodiments called “HM” for objectively measuring nociceptive pain was compared with subject reported pain scoring as ground truths. Visual analog scale (VAS) and numeric rating scale (NRS) were ground truths, respectively, for healthy volunteers and woman in labor. Ranges for VAS and NRS are defined as: 0, 1 to 3, 4-6, 7 to 10, respectively for no pain, mild pain, moderate pain, and severe pain.

Dyspnea: Dyspnea (shortness of breath) monitoring will be a key component of the proposed system. The center for disease control (CDC) guidelines recommends monitoring dyspnea (shortness of breath) as a major indicator of increased criticality in viral pneumonia cases such as COVID-19. The level of dyspnea, or air hunger, does not always correlate with oxygen saturation^{9,10}. Conversely, a viral pneumonia patient can be hypoxemic, even if the patient is not particularly short of breath. For patients with dyspnea but a normal oxygen saturation (SpO₂), supplemental oxygenation will not mitigate the risk of respiratory progression.

Dangers at el.¹¹ reported that the sensation of dyspnea and pain share several characteristics and neural networks in the brain. They also concluded that air hunger interferes with the cortical mechanisms responsible to painful laser skin stimulation. This hypothesis was validated by simulating dyspnea during a CO₂ test (the amount of CO₂ gas in the subject's blood) in the absence of a physical pain stimulus. In this experiment, CO₂, SpO₂, heart rate, systolic & diastolic blood pressures were monitored using standard commercial devices. Similar to the Dangers at el. experiment, the continuously monitored CO₂ rate was controlled to not exceed 50% to avoid a contribution of hypoxia to dyspnea and air hunger. CO₂ was increased twice to trigger dyspnea as shown in FIG. 2 above. This shows SpO₂ and CO₂ rate are not necessarily correlated. This re-validates Dangers at el. findings: “dyspnea and pain are sensations that share several characteristics and neural networks in the brain”. This also confirms that level of dyspnea, or air hunger, does not always correlate with oxygen saturation^{16,17}.

The term “computer” is intended to have a broad meaning that may be used in computing devices such as, e.g., but not limited to, standalone or client or server devices. The computer may be, e.g., (but not limited to) a mobile phone, tablet, personal computer (PC) system running an operating system such as, e.g., (but not limited to) MICROSOFT® WINDOWS® NT/98/2000/XP/Vista/Windows 7/8/etc. available from MICROSOFT® Corporation of Redmond, Wash., U.S.A. or an Apple computer executing MAC® iOS, MAC® OS from Apple® of Cupertino, Calif., U.S.A. However, the invention is not limited to these platforms. Instead, the invention may be implemented on any appropriate computer system running any appropriate operating system. In one illustrative embodiment, the present invention may be implemented on a computer system operating as discussed herein. The computer system may include, e.g., but is not limited to, a main memory, random access memory (RAM), and a secondary memory, etc. Main memory, random access memory (RAM), and a secondary memory, etc., may be a computer-readable medium that may be configured to store instructions configured to implement one or more embodiments and may comprise a random-access memory (RAM) that may include RAM devices, such as Dynamic RAM (DRAM) devices, flash memory devices, Static RAM (SRAM) devices, etc.

The secondary memory may include, for example, (but is not limited to) a hard disk drive and/or a removable storage drive, representing a floppy diskette drive, a magnetic tape drive, an optical disk drive, a compact disk drive CD-ROM, flash memory, etc. The removable storage drive may, e.g., but is not limited to, read from and/or write to a removable storage unit in a well-known manner. The removable storage unit, also called a program storage device or a computer program product, may represent, e.g., but is not limited to, a floppy disk, magnetic tape, optical disk, compact disk, etc. which may be read from and written to the removable storage drive. As will be appreciated, the removable storage unit may include a computer usable storage medium having stored therein computer software and/or data.

In alternative illustrative embodiments, the secondary memory may include other similar devices for allowing computer programs or other instructions to be loaded into the computer system. Such devices may include, for example, a removable storage unit and an interface. Examples of such may include a program cartridge and cartridge interface (such as, e.g., but not limited to, those found in video game devices), a removable memory chip (such as, e.g., but not limited to, an erasable programmable read only memory (EPROM), or programmable read only memory (PROM) and associated socket, and other removable storage units and interfaces, which may allow software and data to be transferred from the removable storage unit to the computer system.

The computer may also include an input device may include any mechanism or combination of mechanisms that may permit information to be input into the computer system from, e.g., a user. The input device may include logic configured to receive information for the computer system from, e.g. a user. Examples of the input device may include, e.g., but not limited to, a mouse, pen-based pointing device, or other pointing device such as a digitizer, a touch sensitive display device, and/or a keyboard or other data entry device (none of which are labeled). Other input devices may include, e.g., but not limited to, a biometric input device, a video source, an audio source, a microphone, a web cam, a video camera, and/or other camera. The input device may communicate with a processor either wired or wirelessly.

The computer may also include output devices which may include any mechanism or combination of mechanisms that may output information from a computer system. An output device may include logic configured to output information from the computer system. Embodiments of output device may include, e.g., but not limited to, display, and display interface, including displays, printers, speakers, cathode ray tubes (CRTs), plasma displays, light-emitting diode (LED) displays, liquid crystal displays (LCDs), printers, vacuum florescent displays (VFDs), surface-conduction electron-emitter displays (SEDs), field emission displays (FEDs), etc. The computer may include input/output (I/O) devices such as, e.g., (but not limited to) communications interface, cable and communications path, etc. These devices may include, e.g., but are not limited to, a network interface card, and/or modems. The output device may communicate with processor either wired or wirelessly. A communications interface may allow software and data to be transferred between the computer system and external devices.

The term “data processor” is intended to have a broad meaning that includes one or more processors, such as, e.g., but not limited to, that are connected to a communication infrastructure (e.g., but not limited to, a communications bus, cross-over bar, interconnect, or network, etc.). The term data processor may include any type of processor, microprocessor and/or processing logic that may interpret and execute instructions (e.g., for example, a field programmable gate array (FPGA)). The data processor may comprise a single device (e.g., for example, a single core) and/or a group of devices (e.g., multi-core). The data processor may include logic configured to execute computer-executable instructions configured to implement one or more embodiments. The instructions may reside in main memory or secondary memory. The data processor may also include multiple independent cores, such as a dual-core processor or a multi-core processor. The data processors may also include one or more graphics processing units (GPU) which may be in the form of a dedicated graphics card, an integrated graphics solution, and/or a hybrid graphics solution. Various illustrative software embodiments may be described in terms of this illustrative computer system. After reading this description, it will become apparent to a person skilled in the relevant art(s) how to implement the invention using other computer systems and/or architectures.

The term “data storage device” is intended to have a broad meaning that includes removable storage drive, a hard disk installed in hard disk drive, flash memories, removable discs, non-removable discs, etc. In addition, it should be noted that various electromagnetic radiation, such as wireless communication, electrical communication carried over an electrically conductive wire (e.g., but not limited to twisted pair, CAT5, etc.) or an optical medium (e.g., but not limited to, optical fiber) and the like may be encoded to carry computer-executable instructions and/or computer data that embodiments of the invention on e.g., a communication network. These computer program products may provide software to the computer system. It should be noted that a computer-readable medium that comprises computer-executable instructions for execution in a processor may be configured to store various embodiments of the present invention.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### Appendix: Validation and Verification

### Description

Some embodiments provide a novel biomarker called “Hm index” to objectively measure nociceptive pain and dyspnea in real-time by utilizing cerebral hemoglobin oxygenation and hemodynamic changes measured using optical spectrometry technology. This biomarker is computed using signal processing and predictive model algorithms and displayed as a single number to quantify pain and dyspnea. The nociceptive pain biomarker algorithm was validated in a three-arm clinical study¹⁸ (n>100): child-birth procedure (OB), general anesthesia or sedated patients (GA), and healthy volunteers. Objective pain was based on assessing changes in hemodynamic response as measured by the system and it demonstrated over 90% correlation with the level of pain experienced by patients/volunteers in the two arms where subjective reporting was possible.

To distinguish pain and dyspnea using Hm index, changes in respiratory vital metrics such as respiration rate and SpO₂ are used. The rise in respiratory rate increases the work of breathing, and the obstructed outflow of air in COPD leads to worsening hyperinflation and heightened stimulation of mechanoreceptors. This results in more dyspnea, which may make the patient breath faster and worsen the hyperinflation. If significant changes are observed in respiratory metrics, Hm index indicates dyspnea and not pain. Otherwise Hm index indicates pain and not dyspnea. In other words, for pain, dyspnea is a false positive and for dyspnea pain is a false positive. By observing changes in respiratory metrics, what Hm index indicates for can be identified, and false positives eliminated. Multiple cardiovascular events other than dyspnea could cause changes in respiratory metrics and they alone cannot be used as biomarkers for dyspnea, however these metrics along with Hm index along with these metrics could precisely quantify dyspnea.

### System

The system 100 of FIG. 1 is capable of continuously measuring vital signs, critical health functions (SpO₂/hypoxia, respiration rate, skin impedance, bio-potentials including ECG, heart rate, blood oxygenation, blood deoxygenation, body temperature, body posture and activities, dyspnea), and real-time objective pain (nociception). The system consists of a small wearable patch 105, a tablet/smart phone-based app 115, and a cloud-based data hub 120 (see FIG. 1). The patch includes a multi-frequency LED, multiple optical detectors, multiple impedance sensors, electrodes, an accelerometer/gyroscope, a battery, a temperature sensor and a Bluetooth transmitter. It can be used in both a wired and wireless format. In this example the patch 15 is worn on the forehead, but in other embodiments it may be worn anywhere on the head, such as the neck or the car.

### Data Collection

Multiple data using the remote monitoring device 100 (wearable and an IOS app) were collected from several subjects for end-to-end validation and verification and make sure it operates as expected. Simultaneously data was collected from Masimo's MightySat® Fingertip Pulse Oximeter¹⁹ to be used to cross validate and evaluate the performance of the system. Data recording procedure and duration was as following:

A baseline data of at least 2 minutes was recorded from each subject.

Subject was asked to hold it breathes for 30 seconds and then release it.

Subject were asked to breathe to bag for 2 minutes.

Repeat this procedure one more time.

### Results:

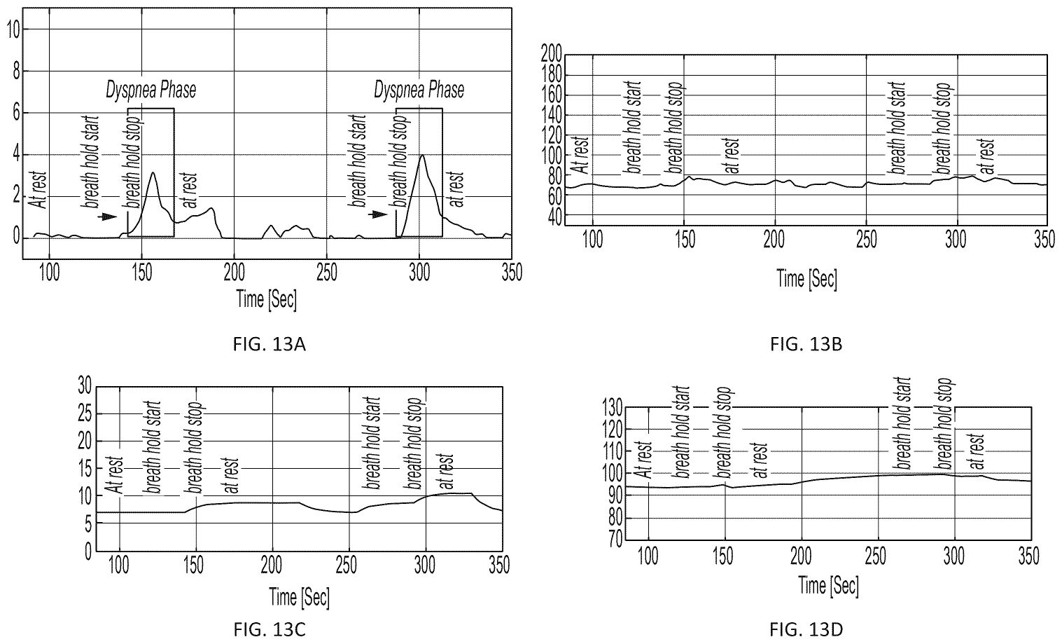

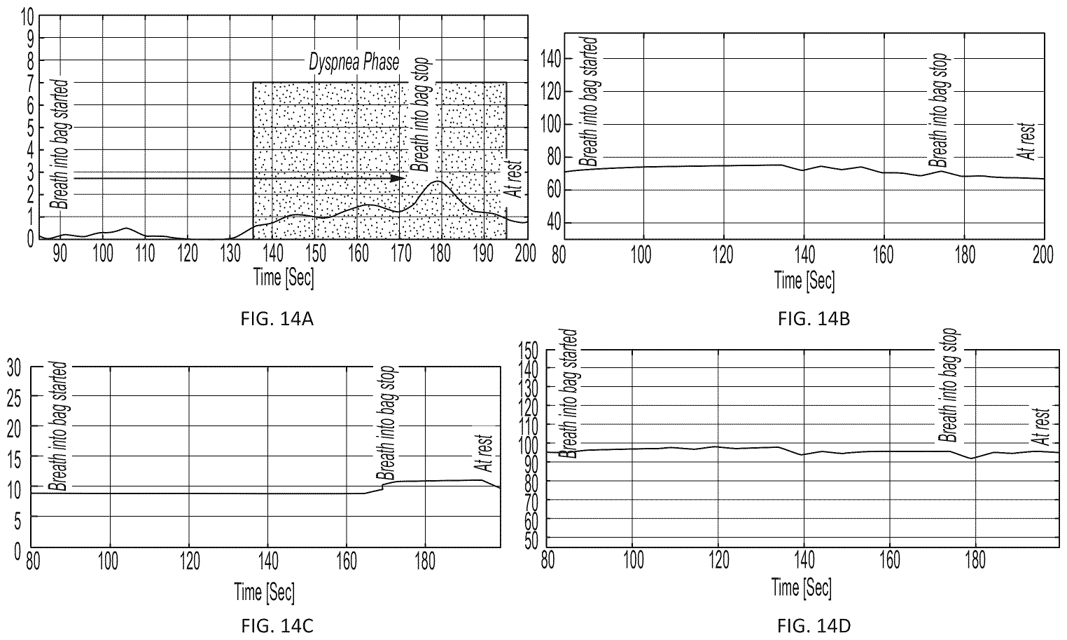

The dyspnea scoring algorithm was validated using two types of data collected from multiple subjects: a) breath hold for 30 seconds to a minute; b) breathing to bag for 2 minutes. FIGS. 13A-13D show routine vital signs (FIG. 13B: heart rate, FIG. 13C: respiration rate, FIG. 13D: SpO₂) and the dyspnea scoring index (FIG. 13A) for a typical data with breath holding every two minutes. As can be seen routine vital signs couldn't clearly reflect dyspnea phases, however the dyspnea scoring index clearly shows and scored dyspnea phases. FIGS. 14A-14D show routine vital signs (FIG. 14B: heart rate, FIG. 14C: respiration rate, FIG. 14D: SpO₂) and the dyspnea scoring index (FIG. 14A) for a typical data with breathing to a bag for two minutes. As can be seen routine vital signs couldn't clearly reflect dyspnea phases, however the dyspnea scoring index clearly shows and scored dyspnea phases.

### REFERENCES

1. World Health Organization (WHO), Coronavirus disease 2019 (COVID-19) Situation Report-46, p. 2
2. Pandharipande P P, Girard T D, Jackson J C, Morandi A, Thompson J L, Pun B T, et al. Long-term cognitive impairment after critical illness. N Engl J Med. 2013; 369 (14): 1306-16.
3. Theodoros Ladopoulos, Maria Giannaki, Christina Alexopoulou, Athanasia Proklou, Emmanuel Pediaditis, Eumorfia Kondili, Ann Gastroenterol. 2018 May-Jun.; 31 (3): 273-281. Published online 2018 Mar. 15. doi: 10.20524/aog.2018.0250
4. Observational Pain Scales in Critically Ill Adults, Critical Care Nurse, June 2013
5. FDA, What's new related to drugs, https://www.fda.gov/drugs/news-events-human-drugs/whats-new-related-drugs
6. Higher Mortality Rate in Ventilated COVID-19 Patients in Large Sample-Medscape-Apr. 13, 2020.
7. Barrett M L, Smith M W, Elixhauser A, Honigman L S, Pines J M. Utilization of intensive care services, 2011. Statistical Brief #185. Healthcare Cost and Utilization Project. Agency for Healthcare Research and Quality. November 2014. http://hcup-us.ahrq.gov/reports/statbriefs/sb185-Hospital-Intensive-Care-Units-2011.jsp. Accessed Jun. 3, 2019.
8. Wunsch H, Wagner J, Herlim M, Chong D H, Kramer A A, Halpern S D. ICU occupancy and mechanical ventilator use in the United States. Crit Care Med. 2013 December; 41 (12): 2712-2719.
9. Gondos T., Szabó V., Sárkány A., Sárkány A. and Halász G. (2016). “Estimation of the severity of breathlessness in the emergency department: a dyspnea score.” BMC Emerg Med.; 17:13.
10. Breathlessness vs. Oxygen Saturation with COPD: Understanding the Difference. https://copd.net/clinical/breathlessness-vs-oxygen-saturation/.Gondos T., Szabó V., Sárkány A., Sárkány A. and Halász G. (2016). “Estimation of the severity of breathlessness in the emergency department: a dyspnea score.” BMC Emerg Med.; 17:13.
11. Dangers L., Laviolette L., Similowski T., and Morélot-Panzini C. (2015). “Interactions Between Dyspnea and the Brain Processing of Nociceptive Stimuli: Experimental Air Hunger Attenuates Laser-Evoked Brain Potentials in Humans.” Front. Physiol., 1 Dec. 2015.
12. Evans K C. Cortico-limbic circuitry and the airways: insights from functional neuroimaging of respiratory afferents and efferents. Biol Psychol 2010; 84:13-25.
13. Rendell Ashton and Dileep Raman, Dyspnea, Disease Management, Cleveland Clinic, Center for Continuing Education, July 2015, http://www.clevelandclinicmeded.com/medicalpubs/diseasemanagement/pulmonary/dyspnea
14. Azevedo-Santos I F, DeSantana J M. Pain measurement techniques: spotlight on mechanically ventilated patients. J Pain Res. 2018; 11:2969-2980. Published 2018 Nov. 21. doi: 10.2147/JPR.S151169.
15. Eisenried A, Austin N, Cobb B, Akhbardeh A, Carvalho B, Ycomans D C, Tzabazis A Z. Correlation of changes in hemodynamic response as measured by cerebral optical spectrometry with subjective pain ratings in volunteers and patients: a prospective cohort study. J Pain Res. 2018 Sep. 24; 11:1991-1998.
16. Lorenz, J., S. Minoshima and K. L. Casey (2003). “Keeping pain out of mind: the role of the dorsolateral prefrontal cortex in pain modulation.” Brain 126 (Pt 5): 1079-1091.
17. Strangman G., B. D. A., and Sutton J. P. (2002). “Non-Invasive Neuroimaging Using Near-Infrared Light.” Society of Biological Psychiatry (52): 679-693.
18. Eisenried A, Austin N, Cobb B, Akhbardeh A, Carvalho B, Yeomans D C, Tzabazis A Z. Correlation of changes in hemodynamic response as measured by cerebral optical spectrometry with subjective pain ratings in volunteers and patients: a prospective cohort study. J Pain Res. 2018 Sep. 24; 11:1991-1998.
19. https://www.masimopersonalhealth.com/products/mightysat-fingertip-pulse-oximeter-with-bluetooth-le-rrp-pvi
20. Sanchez-Morillo D, Fernandez-Granero M A, Leon-Jimenez A. Use of predictive algorithms in-home monitoring of chronic obstructive pulmonary disease and asthma: A systematic review. Chron Respir Dis. 2016; 13 (3): 264-283. doi: 10.1177/1479972316642365.
21. Peng J, Chen C, Zhou M, Xie X, Zhou Y, Luo C H. A Machine-learning Approach to Forecast Aggravation Risk in Patients with Acute Exacerbation of Chronic Obstructive Pulmonary Disease with Clinical Indicators. Sci Rep. 2020 Feb. 20; 10 (1): 3118. doi: 10.1038/s41598-020-60042-1. Erratum in: Sci Rep. 2021 Mar. 2; 11 (1): 5324.
22. Moll M, Qiao D, Regan E A, Hunninghake G M, Make B J, Tal-Singer R, McGeachie M J, Castaldi P J, San Jose Estepar R, Washko G R, Wells J M, LaFon D, Strand M, Bowler R P, Han M K, Vestbo J, Celli B, Calverley P, Crapo J, Silverman E K, Hobbs B D, Cho M H. Machine Learning and Prediction of All-Cause Mortality in COPD. Chest. 2020 September; 158 (3): 952-964. doi: 10.1016/j.chest.2020.02.079. Epub 2020 Apr. 27.
23. F. P. Chmiel, D. K. Burns, J. B. Pickering, Alison Blythin, T. M. A. Wilkinson, M. J. Boniface, Retrospective development and evaluation of prognostic models for exacerbation event prediction in patients with Chronic Obstructive Pulmonary Disease using data self-reported to a digital health application, medRxiv 2020.11.30.20237727; doi: https://doi.org/10.1101/2020.11.30.20237727.

1. A system for quantitatively measuring dyspnea being experienced by a person, comprising:
a hemodynamic sensor system configured to be held in close proximity to, or in contact with, said one of said person's head and said person's neck, said hemodynamic sensor system configured to provide measurements of hemodynamic parameters of said person;
a respiratory sensor system configured to provide measurements of respiratory metrics of said person; and
a data processor configured to communicate with said hemodynamic sensor system to receive said measurements of hemodynamic parameters and configured to communicate with said respiratory sensor system to receive said measurements of respiratory metrics, said data processor being further configured to:
calculate, based on the measurements of hemodynamic parameters, a quantitative score to represent a state of physical discomfort experienced by the person; and
make a determination, based on the quantitative score and the measurements of respiratory metrics, that the state of physical discomfort experienced by the person is caused by dyspnea,
wherein the determination comprises a sub-determination that the respiratory metrics have changed by a particular amount within a particular period of time.

2. The system of claim 1, wherein the quantitative score is calculated using a predictive model algorithm.

3. The system of claim 1, wherein the measurements of hemodynamic parameters comprise a measurement of a change in blood oxygenation.

4. The system of claim 1, wherein the measurements of respiratory metrics comprise a measurement of at least one of a carbon dioxide rate, a respiration rate, and an oxygen saturation percentage.

5. The system of claim 1, wherein the hemodynamic sensor system and the respiratory sensor system each comprise at least one of an optical sensor, an infrared sensor, a near-infrared sensor, surface electrode, and a temperature sensor.

6. The system of claim 1 further comprising a coughing sensor system configured to provide measurements of coughing dynamics of said person, said data processor further configured to communicate with said coughing sensor system to receive said measurements of coughing dynamics, wherein the determination is further based on the measurements of coughing dynamics, said coughing sensor system comprising at least one of an accelerometer, a microphone, and a gyroscope.

7. The system of claim 1 further comprising a vital signs sensor system configured to provide measurements of vital signs of said person, said data processor further configured to communicate with said vital signs sensor system to receive said measurements of vital signs, wherein the determination is further based on the measurements of vital signs, wherein the measurements of vital signs comprise a measurement of at least one of a respiration rate, a heart rate, an impedance, a bio-potential, and a body temperature, said vital signs sensor system comprising at least one of an optical sensor, an infrared sensor, a near-infrared sensor, an electrode and a temperature sensor.

8. The system of claim 1, wherein the data processor is configured to generate, based on the quantitative score and the determination, a treatment plan for the person.

9. A non-transitory machine-readable medium storing a program for quantitatively measuring dyspnea being experienced by a person, which when executed by a data processor configures said data processor to:
receive measurements of hemodynamic parameters of said person, from a hemodynamic sensor system held in close proximity to, or in contact with, one of said person's head and said person's neck;
receive measurements of respiratory metrics of said person from a respiratory sensor system;
calculate, based on the measurements of hemodynamic parameters, a quantitative score to represent a state of physical discomfort experienced by the person; and
make a determination, based on the quantitative score and the measurements of respiratory metrics, that the state of physical discomfort experienced by the person is caused by dyspnea,
wherein the determination comprises a sub-determination that the respiratory metrics have changed by a particular amount within a particular period of time.

10. The machine-readable medium of claim 9, wherein the quantitative score is calculated using a predictive model algorithm.

11. The machine-readable medium of claim 9, wherein the measurements of hemodynamic parameters comprise a measurement of a change in blood oxygenation.

12. The machine-readable medium of claim 9, wherein the measurements of respiratory metrics comprise a measurement of at least one of a carbon dioxide rate, a respiration rate, and an oxygen saturation percentage.

13. The machine-readable medium of claim 9, wherein the hemodynamic sensor system and the respiratory sensor system each comprise at least one of an optical sensor, an infrared sensor, a near-infrared sensor, and a temperature sensor.

14. The machine-readable medium of claim 9, said data processor further configured to receive measurements of coughing dynamics of said person from a coughing sensor system comprising at least one of an accelerometer, a microphone, and a gyroscope, wherein the determination is further based on the measurements of coughing dynamics.

15. The machine-readable medium of claim 9, said data processor further configured to receive measurements of vital signs of said person from a vital signs sensor system comprising at least one of an optical sensor, an infrared sensor, a near-infrared sensor, an electrode and a temperature sensor, wherein the determination is further based on the measurements of vital signs, wherein the measurements of vital signs comprise a measurement of at least one of a heart rate, an impedance, a bio-potential, and a body temperature.

16. The machine-readable medium of claim 9, wherein executing the program further configures the data processor to generate, based on the quantitative score and the determination, a treatment plan for the person.

17. A method for quantitatively measuring dyspnea being experienced by a person, comprising:
receiving measurements of hemodynamic parameters of said person, from a hemodynamic sensor system held in close proximity to, or in contact with, one of said person's head and said person's neck;
receiving measurements of respiratory metrics of said person from a respiratory sensor system;
calculating, based on the measurements of hemodynamic parameters, a quantitative score to represent a state of physical discomfort experienced by the person; and
making a determination, based on the quantitative score and the measurements of respiratory metrics, that the state of physical discomfort experienced by the person is caused by dyspnea,
wherein the determination comprises a sub-determination that the respiratory metrics have changed by a particular amount within a particular period of time.

18. The method of claim 17, wherein the quantitative score is calculated using a predictive model algorithm.

19. The method of claim 17, wherein the measurements of hemodynamic parameters comprise a measurement of a change in blood oxygenation.

20. The method of claim 17, wherein the measurements of respiratory metrics comprise a measurement of at least one of a carbon dioxide rate, a respiration rate, and an oxygen saturation percentage.
